(19) United States
(12) Patent Application Publication
(10) Pub. No.: US 2006/0078529 A1
(43) Pub. Date: Apr. 13, 2006

(54) Hair conditioning composition comprising alkyl diquaternized ammonium salt cationic surfactant

(76) Inventors: Mikio Uchida, Ashiya (JP); Jian-Zhong Yang, Kobe (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/249,616

(22) Filed: Oct. 13, 2005

Related U.S. Application Data
(60) Provisional application No. 60/618,543, filed on Oct. 13, 2004.

(51) Int. Cl. A61K 8/41

(52) U.S. Cl. 424/70.28

(57) ABSTRACT

Disclosed is a hair conditioning composition comprising an alkyl diquaternized ammonium salt cationic surfactant, a high melting point fatty compound, and an aqueous carrier. The composition of the present invention can provide improved conditioning benefits such as smoothness, softness, and ease of combing for both wet hair and dry hair.

### CROSS REFERENCE TO RELATED APPLICATION

This application claims the benefit of U.S. Provisional Application No. 60/618,543, filed on Oct. 13, 2004.

### FIELD OF INVENTION

The present invention relates to a hair conditioning composition comprising an alkyl diquaternized ammonium salt cationic surfactant, a high melting point fatty compound, and an aqueous carrier. The composition of the present invention can provide improved conditioning benefits such as smoothness, softness, and ease of combing for both wet hair and dry hair.

### BACKGROUND OF THE INVENTION

Human hair becomes soiled due to its contact with the surrounding environment and from the sebum secreted by the scalp. The soiling of hair causes it to have a dirty feel and an unattractive appearance. The soiling of the hair necessitates shampooing with frequent regularity.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils and other natural conditioning and moisturizing components. The hair can further be left with increased levels of static upon drying, which can interfere with combing and result in a condition commonly referred to as “fly-away hair”, or contribute to an undesirable phenomena of “split ends”, particularly for long hair.

A variety of approaches have been developed to condition the hair. A common method of providing conditioning benefit to the hair is through the use of hair conditioning agents such as cationic surfactants and polymers, high melting point fatty compounds, low melting point oils, silicone compounds, and mixtures thereof. Most of these conditioning agents are known to provide conditioning benefits by depositing on the hair.

However, it is still not easy to obtain expected conditioning efficacy from the conditioning agents. It is still not easy to obtain expected deposition of the conditioning agents.

Based on the foregoing, there remains a desire for hair conditioning compositions which provide improved conditioning benefits such as smoothness, softness, and ease of combing for both wet hair and dry hair There also remains a desire for hair conditioning compositions which provide improved deposition of conditioning agents.

None of the existing art provides all of the advantages and benefits of the present invention.

### SUMMARY OF THE INVENTION

The present invention is directed to a hair conditioning composition comprising by weight:

(A) from about 0.01% to about 4.0% of a cationic surfactant being an alkyl diquaternized ammonium salt selected from the group consisting of following (A1), (A2), and mixtures thereof:
(A1) an alkyl diquaternized ammonium salt having the formula: 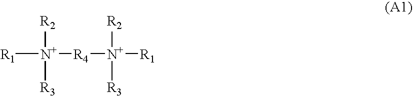
wherein R₁ represents straight or branched, saturated or unsaturated, and/or functionalized or non-functionalized C12-C30 alkyl chain, wherein the functionalized alkyl chain is that containing one or more functional groups selected from the group consisting of —OH, —COO—, —OCO—, —CONH—, —NHCO—, —O—, and —(R₅—O)n- wherein R₅ is C2-4 alkylene and n is integer from 0 to 30; R₂ represents C1-3 alkyl, hydroxyalkyl, or —(R₅—O)n-H; R₃ represents R₂ or H; and R₄ represents functionalized or non-functionalized C2-8 alkylene, wherein the functionalized alkylene is that containing one or more functional groups selected from the group consisting of —OH, —COO—, —OCO—, —CO—NH—, —O—, and —(R₅—O)n-; and
(A2) an alkyl diquaternized ammonium salt having the formula: 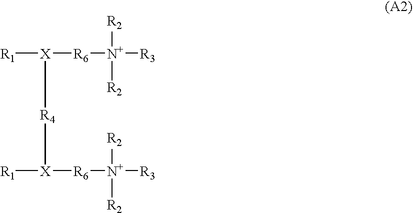
wherein R₁—R₄ represent the same structures as described above; X represents CH or N; and R₆ represents functionalized or non-functionalized C1-10 alkylene wherein the functionalized alkylene is that containing one or more functional groups selected from the group consisting of —OH, —COO—, —OCO—, —CONH—, —NHCO—, —O—, and —(R₅—O)n-;

(B) from about 0.1% to about 20% of a high melting point fatty compound; and

(C) an aqueous carrier.

The hair conditioning composition of the present invention can provide improved conditioning benefits such as smoothness, softness, and ease of combing for both wet hair and dry hair. These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Compositions

The hair conditioning composition of the present invention comprises an alkyl diquaternized ammonium salt cationic surfactant, a high melting point fatty compound, and an aqueous carrier. It is believed that; by the use of the alkyl diquaternized ammonium salt cationic surfactant, the compositions of the present invention provides improved conditioning benefits. It is also believed that such improved conditioning benefits are due to improved deposition of the alkyl diquaternized ammonium salt cationic surfactant and improved robustness of gel matrix, i.e., tighter lamellar gel matrix formed by the alkyl diquaternized ammonium salt cationic surfactant and the fatty compound.

It is believed that, the alkyl diquaternized ammonium salt cationic surfactant, the fatty compound, and aqueous carrier form a gel matrix including a lamellar gel matrix in the composition of the present invention. The gel matrix is suitable for providing various conditioning benefits such as slippery feel and smoothness during the application to wet hair and softness and moisturized feel on dry hair. Among the gel matrix, lamellar gel matrix can provide improved slippery feel during the application to wet hair. Among the lamellar gel matrix, tighter lamellar gel matrix can provide improved slippery feel during the application to wet hair. It is believed that: the use of the alkyl diquaternized ammonium salt cationic surfactant of the present invention results in tighter lamellar gel matrix; and such tighter lamellar gel matrix results in improved conditioning benefits especially slippery feel and smoothness on wet hair.

In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:4.

It has been also found that: the alkyl diquaternized ammonium salt cationic surfactant can provide tighter lamellar gel matrix, compared to tertiary amine, tertiary amine salt, mono-long alkyl quaternized ammonium salt cationic surfactant such as behentrimonium chloride and stearyltrimethylammonium chloride, di-long alkyl quaternized ammonium salt cationic surfactants. Preferably, in view of obtaining tighter lamellar gel matrix, the composition of the present invention is substantially free of other cationic surfactants than those required in the present invention. Such “other cationic surfactant” includes, for example, mono-long alkyl quaternized ammonium salt in which the anion is not C1-C4 alkyl sulfate, tertiary amines, tertiary amine salts, and di-long alkyl quaternized ammonium salts. In the present invention, “substantially free of other cationic surfactants” means that the composition contains 1% or less, preferably 0.5% or less, more preferably totally 0% of total of such other cationic surfactants.

The hair conditioning composition of the present invention is preferably substantially free of anionic compounds, in view of stability of the gel matrix. Anionic compounds herein include anionic surfactants and anionic polymers. In the present invention, “substantially free of anionic compounds” means that the composition contains 1% or less, preferably 0.5% or less, more preferably 0% of anionic compounds.

The hair conditioning composition of the present invention has a pH of preferably from about 3 to about 9, more preferably from about 3 to about 7.

Alkyl Diquaternized Ammonium Salt Cationic Surfactant

The compositions of the present invention comprise an alkyl diquaternized ammonium salt cationic surfactant. The alkyl diquaternized ammonium salt cationic surfactant is contained at a level of from about 0.01% to about 10%, preferably from about 0.01% to about 5.0%, more preferably from about 0.1% to about 3.5%.

The alkyl diquaternized ammonium salt cationic surfactants useful herein are those having two quaternized nitrogen atoms. The alkyl diquaternized ammonium salt cationic surfactants useful herein are those selected from the group consisting of following (A1), (A2), and mixtures thereof:

(A1) an alkyl diquaternized ammonium salt having the formula: 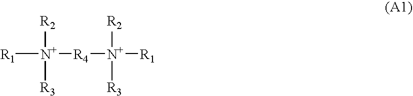
wherein R₁ represents straight or branched, saturated or unsaturated, and/or functionalized or non-functionalized C12-C30 alkyl chain, wherein the functionalized alkyl chain is that containing one or more functional groups selected from the group consisting of —OH, —COO—, —OCO—, —CONH—, —NHCO—, —O—, and —(R₅—O)ₙ— wherein R₅ is C2-4 alkylene and n is integer from 0 to 30; R₂ represents C1-3 alkyl, hydroxyalkyl, or —(R₅—O)ₙ—H; R₃ represents R₂ or H; and R₄ represents functionalized or non-functionalized C2-8 alkylene, wherein the functionalized alkylene is that containing one or more functional groups selected from the group consisting of —OH, —COO—, —OCO—, —CO—NH—, —O—, and —(R₅—O)ₙ—; and

(A2) an alkyl diquaternized ammonium salt having the formula:

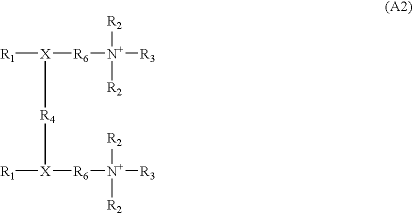

wherein R₁—R₄ represent the same structures as described above; X represents CH or N; and R₆ represents functionalized or non-functionalized C1-10 alkylene wherein the functionalized alkylene is that containing one or more functional groups selected from the group consisting of —OH, —COO—, —OCO—, —CONH—, —NHCO—, —O—, and —(R₅—O)ₙ—.

Preferably, the alkyl diquaternized ammonium salt cationic surfactants are selected from the group consisting of: those having the formula (A1) wherein R₄ is C2-8 alkylene functionalized with a —OH group such as those having an INCI name “Hydroxypropyl-bis-Stearyl-N, N-Dimethylammonium Chloride)”; those having the formula (A2); and mixtures thereof.

High Melting Point Fatty Compound

The hair conditioning composition of the present invention comprises a high melting point fatty compound. The high melting point fatty compound is included in the composition at a level of from about 0.1% to about 20%, preferably from about 1% to about 10%, still more preferably from about 2% to about 8%, by weight of the composition

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

The fatty alcohols useful herein are those having from about 14 to about 30 carbon atoms, preferably from about 16 to about 22 carbon atoms. These fatty alcohols are saturated and can be straight or branched chain alcohols. Nonlimiting examples of fatty alcohols include, cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

Aqueous Carrier

The hair conditioning composition of the present invention comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 95%, preferably from about 30% to about 92%, and more preferably from about 50% to about 90% water.

Silicone Compound

Preferably, the compositions of the present invention preferably contain a silicone compound. It is believed that the silicone compound can provide smoothness and softness on dry hair. The silicone compounds herein are preferably used at levels by weight of the composition of from about 0.1% to about 20%, more preferably from about 0.5% to about 10%, still more preferably from about 1% to about 8%.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

Preferably, the silicone compounds have an average particle size of from about 1 microns to about 50 microns, in the composition.

The silicone compounds useful herein include polyalkyl or polyaryl siloxanes with the following structure: 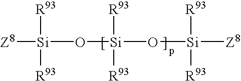
wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and TSF 451 series, and from Dow Corning in their Dow Corning SH200 series.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

The silicone compounds that can be used include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispersibility characteristics of the silicone. These materials are also known as dimethicone copolyols.

Silicone compounds useful herein also include amino substituted materials. Preferred aminosilicones include, for example, those which conform to the general formula (I):
(R₁)ₐG₃₋ₐ-Si—(—OSiG₂)ₙ-(—OSiG_{b}(R₁)_{2-b})ₘ—O—SiG₃₋ₐ(R₁)ₐ
wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH₃)₂ or —NH₂, more preferably —NH₂. Another highly preferred amino silicones are those corresponding to formula (I) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those represented by the following structure: 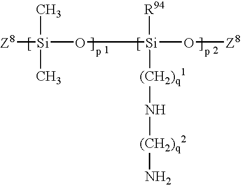
wherein R⁹⁴ is H, CH₃ or OH; p¹ and p² are integers of 1 or above, and wherein sum of p¹ and p² is from 650 to 1,500; q¹ and q² are integers of from 1 to 10. Z⁸ represents groups which block the ends of the silicone chains. Suitable Z⁸ groups include hydroxy, methyl, methoxy, ethoxy, propoxy, and aryloxy. Highly preferred are those known as “amodimethicone”. Commercially available amodimethicones useful herein include, for example, BY16-872 available from Dow Corning.

Other amino substituted silicone polymers which can be used are represented by the formula:

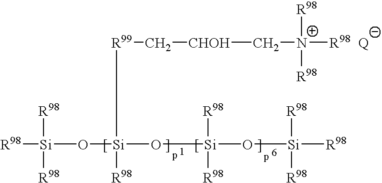

where R⁹⁸ denotes a monovalent hydrocarbon radical having from 1 to 18 carbon atoms, preferably an alkyl or alkenyl radical such as methyl; R⁹⁹ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; p⁵ denotes an average statistical value from 2 to 20, preferably from 2 to 8; p⁶ denotes an average statistical value from 20 to 200, and preferably from 20 to 50.

The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; emollients such as PPG-3 myristyl ether with tradename Varonic APM available from Goldschmidt, Trimethyl pentanol hydroxyethyl ether, PPG-11 stearyl ether with tradename Varonic APS available from Goldschmidt, Stearyl heptanoate with tradename Tegosoft SH available from Goldschmidt, Lactil (mixture of Sodium lactate, Sodium PCA, Glycine, Fructose, Urea, Niacinamide, Inositol, Sodium Benzoate, and Lactic acid) available from Goldschmidt, Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; and sequestering agents, such as disodium ethylenediamine tetra-acetate; ultraviolet and infrared screening and absorbing agents such as octyl salicylate; and antidandruff agents such as zinc pyrithione and salicylic acid.

Polysorbate

The hair conditioning composition of the present invention may contain a polysorbate, in view of adjusting rheology. Preferred polysorbate useful herein includes, for example, polysorbate-20, polysorbate-21, polysorbate-40, polysorbate-60, and mixtures thereof. Highly preferred is polysorbate-20.

The polysorbate can be contained in the composition at a level by weight of preferably from about 0.01% to about 5%, more preferably from about 0.05% to about 2%.

Polypropylene Glycol

Polypropylene glycol useful herein are those having a weight average molecular weight of from about 200 g/mol to about 100,000 g/mol, preferably from about 1,000 g/mol to about 60,000 g/mol. Without intending to be limited by theory, it is believed that the polypropylene glycol herein deposits onto, or is absorbed into hair to act as a moisturizer buffer, and/or provides one or more other desirable hair conditioning benefits.

The polypropylene glycol useful herein may be either water-soluble, water-insoluble, or may have a limited solubility in water, depending upon the degree of polymerization and whether other moieties are attached thereto. The desired solubility of the polypropylene glycol in water will depend in large part upon the form (e.g., leave-on, or rinse-off form) of the hair care composition. For example, a rinse-off hair care composition, it is preferred that the polypropylene glycol herein has a solubility in water at 25° C. of less than about 1 g/100 g water, more preferably a solubility in water of less than about 0.5 g/100 g water, and even more preferably a solubility in water of less than about 0.1 g/100 g water.

The polypropylene glycol can be included in the hair conditioning composition of the present invention at a level of, preferably from about 0.01% to about 10%, more preferably from about 0.05% to about 6%, still more preferably from about 0.1% to about 3% by weight of the composition.

Low Melting Point Oil

Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof. Preferred low melting point oils herein are selected from the group consisting of: ester oils such as pentaerythritol ester oils, trimethylol ester oils, citrate ester oils, and glyceryl ester oils; poly α-olefin oils; and mixtures thereof.

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Particularly useful citrate ester oils herein include triisocetyl citrate with tradename CITMOL 316 available from Bernel, triisostearyl citrate with tradename PELEMOL TISC available from Phoenix, and trioctyldodecyl citrate with tradename CITMOL 320 available from Bernel.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Exxon Mobil Co.

Cationic Polymer

Cationic polymers useful herein are those having an average molecular weight of at least about 5,000, typically from about 10,000 to about 10 million, preferably from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

Polyethylene Glycol

Polyethylene glycol can also be used as an additional component. The polyethylene glycols useful herein that are especially preferred are PEG-2M wherein n has an average value of about 2,000 (PEG-2M is also known as Polyox WSR® N-10 from Union Carbide and as PEG-2,000); PEG-5M wherein n has an average value of about 5,000 (PEG-5M is also known as Polyox WSR® N-35 and as Polyox WSR® N-80, both from Union Carbide and as PEG-5,000 and Polyethylene Glycol 300,000); PEG-7M wherein n has an average value of about 7,000 (PEG-7M is also known as Polyox WSR® N-750 from Union Carbide); PEG-9M wherein n has an average value of about 9,000 (PEG-9M is also known as Polyox WSR® N-3333 from Union Carbide); and PEG-14M wherein n has an average value of about 14,000 (PEG-14M is also known as Polyox WSR® N-3000 from Union Carbide).

Product Forms

The hair conditioning compositions of the present invention can be in the form of rinse-off products or leave-on products, can be opaque, and can be formulated in a wide variety of product forms, including but not limited to creams, gels, emulsions, mousses and sprays. Preferably, the hair composition of the present invention is in the form of rinse-off products. Such rinse-off hair conditioning compositions are preferably used by following steps:
(i) after shampooing hair, applying to the hair an effective amount of the conditioning composition of claim 1 for conditioning the hair; and
(ii) then rinsing the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

All percentages are based upon the total weight of the compositions of the present invention, unless otherwise specified. All such weights as they pertain to listed ingredients are based on the active level and, therefore, do not include carriers or by-products that may be included in commercially available materials.

Compositions (wt %)

| Components | Ex. 1 | Ex. 2 | Ex. 3 | Ex. 4 | Ex. 5 |
|---|---|---|---|---|---|
| Alkyl diquaternized ammonium salt-1 *1 | 2.25 | 1.10 | 1.50 | 1.00 | 3.38 |
| Alkyl diquaternized ammonium salt-2 *2 | — | — | — | 1.00 | — |
| Alkyl diquaternized ammonium salt-3 *3 | — | — | — | — | — |
| Alkyl diquaternized ammonium salt-4 *4 | — | — | — | — | — |
| Cetyl alcohol | 1.86 | 0.80 | 1.25 | 2.32 | 2.32 |
| Stearyl alcohol | 4.64 | 2.30 | 3.10 | 4.18 | 4.18 |
| Silicone compound-1 *6 | — | 4.2 | — | — | — |
| Silicone compound-2 *7 | — | — | 4.2 | — | — |
| Silicone compound-3 *8 | 3.5 | — | — | 3.5 | — |
| Silicone compound-4 *9 | — | — | — | — | 4.2 |
| PPG-34 *10 | — | — | 0.5 | — | — |
| Poly-α-olefin oil *11 | — | — | — | 1.0 | — |
| Benzyl alcohol | 0.4 | 0.4 | 0.4 | 0.4 | 0.4 |
| Methylchloroisothiazolinone/ | 0.0005 | 0.0005 | 0.0005 | 0.0005 | 0.0005 |
| Methylisothiazolinone *12 | | | | | |
| Perfume | 0.35 | 0.35 | 0.5 | 0.25 | 0.5 |
| NaOH | 0.014 | 0.014 | — | 0.014 | — |
| Panthenol *13 | — | 0.05 | 0.05 | 0.05 | — |
| Panthenyl ethyl ether *14 | — | 0.05 | 0.05 | 0.05 | — |
| Hydrolyzed collagen *15 | — | — | 0.01 | 0.01 | 0.01 |
| Vitamin E *16 | — | — | 0.01 | 0.01 | 0.01 |
| Octyl methoxycinnamate | — | — | — | 0.09 | — |
| Benzophenone-3 | — | — | — | 0.09 | — |
| Disodium EDTA | 0.127 | 0.127 | 0.127 | 0.127 | 0.127 |

| | |
|---|---|
| Deionized Water | q.s. to 100% |

| Components | Ex. 6 | Ex. 7 | Ex. 8 | Ex. 9 | Ex. 10 |
|---|---|---|---|---|---|
| Alkyl diquaternized ammonium salt-1 *1 | — | — | — | 0.50 | 1.50 |
| Alkyl diquaternized ammonium salt-2 *2 | 2.50 | | — | — | — |
| Alkyl diquaternized ammonium salt-3 *3 | — | 2.00 | — | — | — |
| Alkyl diquaternized ammonium salt-4 *4 | — | — | 1.50 | — | — |
| Behenyl trimethyl ammonium | — | — | — | 1.25 | 0.50 |
| methosulfate *5 | | | | | |
| Cetyl alcohol | 2.50 | 2.32 | 2.32 | 2.32 | 2.32 |
| Stearyl alcohol | 4.50 | 4.18 | 4.18 | 4.18 | 4.18 |
| Silicone compound-1 *6 | — | — | — | — | 4.2 |
| Silicone compound-2 *7 | — | 2.0 | — | — | — |
| Silicone compound-3 *8 | 3.5 | — | 3.5 | 2.0 | — |
| Silicone compound-4 *9 | — | 2.0 | 0.5 | — | — |
| Polysorbate-20 | 0.2 | — | — | 0.1 | — |
| PPG-34 *10 | — | 0.5 | — | — | — |
| Poly-α-olefin oil *11 | — | — | — | 1.0 | — |
| Benzyl alcohol | 0.4 | 0.4 | 0.4 | 0.4 | 0.4 |
| Methylchloroisothiazolinone/ | 0.0005 | 0.0005 | 0.0005 | 0.0005 | 0.0005 |
| Methylisothiazolinone *12 | | | | | |
| Perfume | 0.5 | 0.5 | 0.5 | 0.5 | 0.5 |
| NaOH | 0.014 | 0.014 | 0.014 | 0.014 | 0.014 |
| Panthenol *13 | 0.05 | 0.05 | 0.05 | 0.05 | — |
| Panthenyl ethyl ether *14 | 0.05 | 0.05 | 0.05 | 0.05 | — |
| Hydrolyzed collagen *15 | — | 0.01 | — | — | — |
| Vitamin E *16 | — | 0.01 | — | — | — |
| Octyl methoxycinnamate | — | 0.09 | — | — | — |
| Benzophenone-3 | — | 0.09 | — | — | — |
| Disodium EDTA | 0.127 | 0.127 | 0.127 | 0.127 | 0.127 |

| Deionized Water | q.s. to 100% |
|---|---|
| Definitions of Components | |
| *1 Alkyl diquaternized ammonium salt-1: Hydroxypropyl-bis-stearyl-N,N-dimethylammonium chloride available from Toho Kagaku K.K. | |
| *2 Alkyl diquaternized ammonium salt-2: Hydroxypropyl-bis-behenyl-N,N-dimethylammonium chloride available from Toho Kagaku K.K. | |
| *3 Alkyl diquaternized ammonium salt-3: Hydroxypropyl-bis-stearamidopropyl-N,N-dimethylammonium chloride available from Toho Kagaku K.K. | |
| *4 Alkyl diquaternized ammonium salt-4: Hydroxypropyl-bis-lauryl-N,N-dimethylammonium chloride available from Toho Kagaku K.K. | |
| *5 Behenyl trimethyl ammonium methosulfate: available from Croda | |
| *6 Silicone compound-1: Dimethicone/Cyclomethicone: a blend dimethicone having a viscosity of 18,000,000 mPa · s and cyclopentasiloxane available from GE Toshiba | |
| *7 Silicone compound-2: Dimethicone blend: a blend of dimethicone having a viscosity of 18,000,000 mPa · s and dimethicone having a viscosity of 200 mPa · s available from GE Toshiba | |
| *8 Silicone compound-3: Terminal aminosilicone which is available from GE having a viscosity 10,000 mPa · s, and having following formula (I): (R₁)ₐG₃₋₁-Si—(—OSiG₂)ₙ-(-OSiG_{b}(R₁)_{2-b})ₘ-O—SiG₃₋ₐ(R₁)ₐ (I) | |
| # wherein G is methyl; a is an integer of 1; b is 0, 1 or 2, preferably 1; n is a number from 400 to about 600; m is an integer of 0; R₁ is a monovalent radical conforming to the general formula CqH_{2q}L, wherein q is an integer of 3 and L is —NH₂ | |

*9 Silicone compound-4: Available from Wacker with a tradename AD1100 and having a viscosity of 4,000 mm² · s⁻¹
*10 PPG-34: New Pol PP-2000 available from Sanyo Kasei
*11 Poly-α-olefin oil: Puresyn 100 available from Exxon Mobil
*12 Methylchloroisothiazolinone/Methylisothiazolinone: Kathon CG available from Rohm & Haas
*13 Panthenol: Available from Roche
*14 Panthenyl ethyl ether: Available from Roche
*15 Hydrolyzed collagen: Peptein 2000 available from Hormel
*16 Vitamin E: Emix-d available from Eisai
*17 Behenamidopropyl dimethylamine: IncromineBB available from Croda

Method of Preparation

The hair conditioning compositions of “Ex. 1” through “Ex. 10” as shown above can be prepared by any conventional method well known in the art. They are suitably made as follows:

Deionized water is heated to 85° C. Cationic surfactants and high melting point fatty compounds are mixed into the water. The water is maintained at a temperature of about 85° C. until the components are homogenized, and no solids are observed. The mixture is then cooled to about 55° C. and maintained at this temperature, to form a gel matrix. Silicone compound is added to the gel matrix. When included, poly α-olefin oils, polypropylene glycols, and/or polysorbates are also added to the gel matrix. The gel matrix is maintained at about 50° C. during this time with constant stirring to assure homogenization. After it is homogenized, it is cooled to room temperature. When included, other additional components such as perfumes and preservatives are added with agitation. A triblender and/or mill can be used in each step, if necessary to disperse the materials.

The embodiments disclosed and represented by the previous “Ex. 1” through “Ex. 10” have many advantages. For example, they are especially suitable for rinse-off products, and can provide improved conditioning benefits such as smoothness, softness, and ease of combing for both wet hair and dry hair.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioning composition comprising by weight:
(A) from about 0.01% to about 10% of a cationic surfactant being an alkyl diquaternized ammonium salt selected from the group consisting of following (A1), (A2), and mixtures thereof:
(A1) an alkyl diquaternized ammonium salt having the formula: 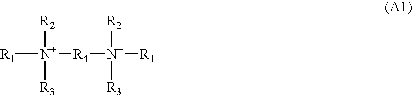
wherein R₁ represents straight or branched, saturated or unsaturated, and/or functionalized or non-functionalized C12-C30 alkyl chain, wherein the functionalized alkyl chain is that containing one or more functional groups selected from the group consisting of —OH, —COO—, —OCO—, —CONH—, —NHCO—, —O—, and —(R₅—O)n- wherein R₅ is C2-4 alkylene and n is integer from 0 to 30; R₂ represents C1-3 alkyl, hydroxyalkyl, or —(R₅—O)n—H; R₃ represents R₂ or H; and R₄ represents functionalized or non-functionalized C2-8 alkylene, wherein the functionalized alkylene is that containing one or more functional groups selected from the group consisting of —OH, —COO—, —OCO—, —CO—NH—, —O—, and —(R₅—O)n-; and
(A2) an alkyl diquaternized ammonium salt having the formula: 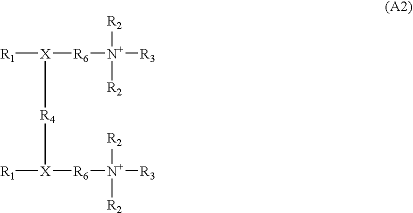
wherein R₁—R₄ represent the same structures as described above; X represents CH or N; and R₆ represents functionalized or non-functionalized C1-10 alkylene
wherein the functionalized alkylene is that containing one or more functional groups selected from the group consisting of —OH, —COO—, —OCO—, —CONH—, —NHCO—, —O—, and —(R₅—O)n-;
(B) from about 0.1% to about 20% of a high melting point fatty compound; and
(C) an aqueous carrier.

2. The hair conditioning composition comprising of claim 1 wherein the alkyl diquaternized ammonium salt cationic surfactant is selected from that having the formula (A1) wherein R₄ represents C2-8 alkylene functionalized with —OH.

3. The hair conditioning composition of claim 1 wherein the alkyl diquaternized ammonium salt cationic surfactant is selected from that having the formula (A2).

4. The hair conditioning composition of claim 1, wherein the composition is substantially free of other cationic surfactants than the alkyl diquaternized ammonium salt cationic surfactant.

5. The hair conditioning composition of claim 1 wherein the weight ratio of the alkyl diquaternized ammonium salt cationic surfactant to the high melting point fatty compound is in the range of from about 1:1 to about 1:10.

6. The hair conditioning composition of claim 1 wherein the weight ratio of the cationic surfactant and the high melting point fatty compound is within the range of from about 1:1 to about 1:4.

7. The hair conditioning composition of claim 1 further comprising from about 0.1% to about 20% of a silicone compound.

8. The hair conditioning composition of claim 7 wherein the silicone compound is an aminosilicone having the general formula:
(R₁)ₐG₃₋ₐ-Si—(—OSiG₂)ₙ-(—OSiG_{b}(R₁)_{2-b})ₘ—O—SiG₃₋ₐ(R₁)ₐ
wherein G is hydrogen, phenyl, hydroxy, or C₁-C₈ alkyl; a is 0 or an integer having a value from 1 to 3; n is a number from 0 to 1,999; m is an integer from 0 to 1,999; the sum of n and m is a number from 1 to 2,000; a and m are not both 0; R₁ is a monovalent radical conforming to the general formula C_{q}H_{2q}L, wherein q is an integer having a value from 2 to 8 and L is selected from the following groups: —N(R₂)CH₂—CH₂—N(R₂)₂;—N(R₂)₂;—N(R₂)₃A⁻; —N(R₂)CH₂—CH₂—NR₂H₂A⁻; wherein R₂ is hydrogen, phenyl, benzyl, or a saturated hydrocarbon radical; A⁻ is a halide ion.

9. The hair conditioning composition of claim 8 wherein the aminosilicone is mixed with a solvent having a low viscosity selected from the group consisting of non-polar, volatile hydrocarbons, volatile cyclic silicones, non-volatile linear silicones, and mixtures thereof.

10. The hair conditioning composition of claim 7 wherein the silicone compound is a mixture of (i) a first silicone having a viscosity of from about 100,000 mPa·s to about 30,000,000 mPa·s at 25° C. and (ii) a second silicone having a viscosity of from about 5 mPa·s to about 10,000 mPa·s at 25° C.

11. The hair conditioning composition of claim 1 wherein the composition is substantially free of anionic surfactants and anionic polymers.

12. A method of conditioning hair, the method comprising following steps:
(i) after shampooing hair, applying to the hair an effective amount of the conditioning composition of claim 1 for conditioning the hair; and
(ii) then rinsing the hair.
